(19) United States
(12) Patent Application Publication
(10) Pub. No.: US 2026/0146956 A1
(43) Pub. Date: May 28, 2026

(54) DARK-FIELD IMAGING SYSTEM

(71) Applicant: TECAN TRADING AG, Mannedorf (CH)

(72) Inventors: Patrick KINNEY, Hayward, CA (US); Philipp OTT, Steg (CH)

(21) Appl. No.: 18/705,704

(22) PCT Filed: Nov. 8, 2021
(86) PCT No.: PCT/US2021/058376

§ 371 Date: Apr. 29, 2024

(51) Int. Cl. G01N 21/84; G01N 21/47; G01N 33/483; G02B 21/00; G02B 21/10

(52) U.S. Cl. CPC G01N 21/84; G01N 21/4738; G01N 33/4833; G02B 21/10; G02B 21/0004

(57) ABSTRACT

A dark-field imaging system has a platform for holding biologic tissue or cells constituting a plane forming two half-spaces. A light source generates an incident light targeting the biologic tissue or cells. A camera detects diffuse reflection off the biologic tissue or cells. The light source is placed at a first angle relative to the plane, and the camera is placed at a second angle relative to the plane so that the light source, the camera, and the biologic tissue or cells are arranged in one of the two half-spaces formed by the plane. The incident light that is not reflected by the biologic tissue or cells reaches the platform and has a specularly reflecting material that reflects the incident light at least in part by specular reflection.

### TECHNICAL FIELD

The present invention relates generally to a dark-field imaging system and methods for taking high contrast pictures of biological samples or living cells. Specifically, the invention relates to a dark-field imaging system and methods for imaging tissue samples in an application for interacting with areas of interest of biological samples.

### BACKGROUND OF THE INVENTION

Dark-field imaging systems are typically used in microscopy (dark-field microscopy or dark-ground microscopy) and are well suited for uses involving live and unstained biological samples, e.g., unstained formalin fixed paraffin embedded (FFPE) tissue samples. It can be used in both light and electron microscopy. As it excludes the unscattered beam from the image, dark-field imaging produces a picture with a dark background around the specimen. In some cases it is desirable to harvest a small precisely defined portion of a tissue or cell population on a microscope slide, a cell culture chamber, flask, dish, or microplate for further analyses. These analyses may serve the purpose of providing a histopathological diagnosis for taking further therapeutic decisions. Advanced techniques in Biochemistry and Molecular Biology allow this information to be used for highly effective personalized medicine. Formalin fixed paraffin embedded (FFPE) patient tissue for example can be deparaffinized and stained with markers for a wide variety of traits. When such a diagnostic procedure reveals a characteristic or abnormality in a specific area of the tissue, the so-called area of interest (AOI), it may be important to be able to isolate cells from the AOI and extract their nucleic acids, proteins, other subcellular components and/or molecules for further identification. The harvested cells can for example serve as the material for the sequencing of known tumor risk genes. The one or more mutations found will determine the choice of treatment, which could be either a drug or drug combination and/or could be irradiation of the affected tissue or organ. When interacting with areas of interest of biological samples it is important to get a high contrast image of a reference tissue slice or the specimen of interest itself. The latter, often unstained, are best imaged in a dark field configuration.

U.S. Pat. No. 10,876,933 B2 addresses an instrument for automatically dissecting a locally restricted area of interest of a prepared biological sample on a microscope slide, for example an area of interest of a tissue section embedded in paraffin, in an automated manner as a preparation for subsequent analysis steps. The serial sample slides are aligned with the reference slide.

Optical alignment can be done through a backdrop assembly, enabling the users to optimize their slide image by selecting the most appropriate backdrop color based on the type of slide on the stage. This optimizes the contrast of the reference slide image and allows the alignment of the serial sample slides with the reference slide by pattern matching using the outline contours.

### SUMMARY OF THE INVENTION

One limitation of dark-field imaging is the low light levels seen in the final image. Therefore, the specimen must be strongly illuminated, which in the systems known in the state-of-the-art cause substantial light scattering behind the sample including the backdrop assembly.

This diffusely reflected light enters the camera and causes the background around the specimen to not be completely dark. Such a greyish background reduces the contrast of the image and thereby the degree of details that may be recorded.

It is thus an object of the present invention to provide for a dark-field imaging system that provides more image detail in the grayscale range.

This is solved by a dark-field imaging system according to claim 1, which comprises a platform that reflects incident light at least in part by specular reflection. Further favorable embodiments can, for example, be derived from the respective dependent claims.

The dark-field imaging system according to the invention comprises a platform for holding biologic tissue or cells, constituting a plane forming two half-spaces. The dark-field imaging system further comprises a light source, which generates an incident light targeting the biologic tissue or cells, and a camera to detect diffuse reflection off the biologic tissue or cells. The light source is placed at a first angle (a) relative to the plane, and the camera is placed at a second angle (B) relative to the plane, wherein the light source, the camera, and the biologic tissue or cells are arranged in the same one of the two half-spaces formed by the plane. The incident light that is not reflected by the biologic tissue or cells reaches the platform, which comprises a specularly reflecting material that reflects the incident light at least in part by specular reflection.

The presence of a platform that reflects the incident light of the light source at least in part by specular reflection, has the advantage of eliminating diffuse reflections that in other systems occur behind the sample to be analyzed on a microscope slide, cell culture chamber, flask, dish, or microplate. This results in a black or nearly black background around the specimen and allows the camera to capture an image with a significantly improved contrast. In addition, the diffuse light scattered away from the camera is specularly reflected into the camera, resulting in a significant increase in the brightness of the sample to be analyzed. This further ameliorates the contrast of the image.

Specular reflection according to the invention is the unscattered mirror-like reflection of light or other waves or particles, such that an incident light or radiation is reflected at just one angle. A complete specular reflection without any diffuse reflection can only be observed with a few materials including metals that do not allow light to enter, glass, and transparent plastics that have a liquid-like amorphous microscopic structure. While a smooth surface is required for specular reflection, it does not prevent diffuse reflection. Amorphous or non-crystalline solids (e.g., glass, transparent plastics, polymers) produce specular reflection because they don't have any internal subdivisions which are needed for a scattering below the surface. Except for polished aluminum or silver, which are usually used in mirrors and can reflect light specularly with high efficiency, common materials only show a few percent specular reflection, even when perfectly polished and therefore mirror-like. Diffuse reflection according to the invention is the reflection of light or other waves or particles from a surface that undergoes scattering, such that an incident light or radiation is scattered at many angles on the surface of an object. An ideal diffuse reflecting surface is said to exhibit Lambertian reflection, meaning that there is equal luminance when viewed from all directions lying in the half-space adjacent to the surface.

Depending on the material and surface roughness, reflection may be mostly specular, mostly diffuse, or anywhere in between. Many common materials including biologic tissue or cells exhibit a mixture of specular and diffuse reflection. While surface roughness plays a role in diffuse reflection, most of the diffuse reflection comes from scattering centers beneath the surface. When an incident light hits a biologic tissue or a cell with their membranes and their complex internal structure, an impinging ray is partially reflected at a molecular structure while entering in it, is again reflected at an interface to a second molecular structure, enters in it, impinges on the third, and so on. This generates a series of “primary” scattered rays in random directions, which, in turn, through the same mechanism, generate a large number of “secondary” scattered rays, which generate “tertiary” rays, and so forth. It is this diffusely scattered light at each interface, inhomogeneity or imperfection that deviates, reflects or scatters light, that forms the image of the object in the observer's eye or the camera.

According to the invention said platform holds the specimen to be looked at. The platform according to the invention may comprise a stage and a carrier. The specimen, which can be biologic tissue or cells, is usually placed on a carrier. The carrier may be positioned on the stage. If the stage is more or less horizontal, such that the carrier doesn't slide, the carrier will stay in place without being held. According to an embodiment, the carrier may be reversibly attached to the stage, allowing the stage to be arranged at any angle relative to the ground, i.e., as an inclined plane. When the stage is arranged essentially horizontal, instead of placing the carrier with the specimen on top, it may be placed underneath, but in this case the carrier needs to be fastened to the stage.

The platform according to the invention comprise a material that reflects the incident light at least in part by specular reflection. According to an embodiment, the platform comprises a material that reflects the incident light mostly in a specular reflection, i.e., more than 50% of the light from the light source is reflected in a specular reflection, while there is less than 50% of the light reflected in a diffuse reflection. According to an embodiment, the platform comprises a material that reflects at least 90% of the incident light reaching the platform by specular reflection. For the material to reflect incident light mostly in a specular reflection, it must have a smooth mirror-like surface.

According to an embodiment, the platform comprises a metal. When polished and therefore mirror-like, some metals reflect light mostly in a specular reflection., e.g., polished aluminum, silver, or chromium. In an embodiment, the platform comprises aluminum, silver, chromium, gold, or any other metal, or comprises an alloy such as stainless steel. In an embodiment, the platform comprises mirror-like hard chromium.

According to an embodiment, the platform comprises glass, a transparent plastic, or ceramics.

The material reflecting the incident light at least in part by specular reflection may form the surface of the platform. This may be in the form of a block or a layer, wherein a thin coating may be sufficient. In an embodiment in which it forms part of the stage, it faces the carrier with biologic tissue or cells. The material may be an integral part of the stage or may be a layer on the surface of the stage. In an embodiment, the stage is covered with a layer of material that reflects the incident light at least in part by specular reflection.

According to an embodiment, the material specularly reflecting the incident light may be an integral part of the carrier or form a surface layer of the carrier. The material layer on the surface may face the biologic tissue or cells. Alternatively it may form a layer facing the stage. Such a carrier according to the invention may be a microscope slide covered with a metal layer, forming a mirrored glass slide, whereas the metal layer may be on either side, or both sides.

According to an embodiment, the material specularly reflecting the incident light may be resistant to scratching or any other damage, including being resistant to cleaning agents as for example disinfectants. This may be important as a smooth surface is required for specular reflection.

According to an embodiment, the platform comprises a heating/cooling unit. The heating/cooling unit may raise the surface temperature of the stage and thereby may heat the carrier to a temperature which causes the paraffin of FFPE tissue to melt.

In an embodiment the stage is constructed as a temperature adapter in physical contact with the carrier holding biologic tissue or cells, which channels and conducts the temperature from the heating/cooling unit to the one or more carriers with biologic tissue or cells. The temperature of the stage can thereby be elevated to melt the paraffin of formalin fixed paraffin embedded (FFPE) tissue samples and as a result, the light scattering around the biologic tissue or cells is mostly eliminated.

According to an embodiment, the material specularly reflecting the incident light may be conducting temperature such that the temperature from the heating/cooling unit gets transferred to the biologic tissue or cells. This may be important for melting paraffin of formalin fixed paraffin embedded (FFPE) tissue samples, which further reduces light scattering around the specimen, resulting in even more contrast and improved image quality.

According to an embodiment, the heating/cooling unit may lower the surface temperature of the stage and thereby may cool the carrier to a temperature which causes the biologic tissue or cells to freeze or stay frozen. Such freezing may be applied to fresh frozen tissue, in which case the area of interest (AOI) may be removed in a mechanical manner.

According to an embodiment the temperature adapter has a three-dimensional shape which allows the accurate positioning of a rack comprising one or more carriers, e.g., a rack holding several standard microscope slides with FFPE tissue samples arranged on them.

Biologic tissue or cells according to the invention may be any living or dead biologic material, e.g., tissue samples or slices, plated cells, primary and secondary cell cultures. A common method for preserving proteins and vital structures in biopsy specimens involves fixing tissue in formaldehyde, also known as formalin. Formalin-Fixed Paraffin-Embedded (FFPE) tissue specimens are the basis for many research and therapeutic applications including examination, experimental research, diagnostics, and drug development. FFPE tissue is usually prepared on microscope glass slides, allowing interacting with areas of interest of biological samples. Unstained FFPE tissue samples may be imaged in a dark field configuration.

Hardened paraffin however reflects incident light in a scattered diffuse reflection, causing the image to be of low contrast with a greyish background. FFPE tissue specimens are therefore best heated above the melting point of paraffin, which shows much lower light scattering when in a liquid state. As a result, FFPE tissue may be imaged with dark background and high contrast. Glass slides can also be the carrier of choice for other applications, e.g., fluorescent microscopy of fixed tissue culture cells. Living cells on the other hand may have to be grown in cell culture chambers, flasks, dishes, or microplates, which may be of any material or combination of materials.

The carrier according to the invention may be a microscope slide, a cell culture chamber, flask, dish, or microplate. Besides holding fixed tissue and cells, a cell culture chamber, flask, dish, or microplate can hold live cells. In an embodiment, the carrier is made of a material transparent for the wavelength emitted by the light source. The carrier according to the invention may comprise or consist of glass, polystyrene, tissue culture polystyrene, polypropylene, cyclic olefin copolymer, polymethylmethacrylate, or any other material transparent for the wavelength emitted by the light source.

According to an embodiment, the stage comprises means for holding or reversibly attaching one or more carriers, i.e., a fastener. Such a fastener according to the invention may comprise for example a clip, a spring, a clamp, a screw, form closure, a frame or recess, a bayonet lock, a screwed joint, adhesives, Velcro®, or any other fastening element known in the art. Preferred means for holding or reversibly attaching one or more carriers can easily be detached without destruction. Means for holding or reversibly attaching one or more carriers allow the operator to place the one or more carriers in a predetermined position and to move the stage without shifting the one or more carriers. As an example, a recess with the dimensions of a microscope slide is a simple way to hold a microscope slide in place without any further means for holding or reversibly attaching the slide. When the stage or the entire dark-field imaging system according to the invention is placed at an angle relative to the horizon, or upside down, the one or more carriers with biologic tissue or cells need to be held in place by means for holding or reversibly attaching one or more carriers that are independent of gravitational forces, comprising such as a clip, a spring, a clamp, a screw, form closure, a bayonet lock, a screwed joint, Velcro®, or adhesives. In an embodiment, the platform comprises means for holding or reversibly attaching one or more carriers in the form of a rack, which holds one or more carriers and can be detached from the rest of the platform together with the one or more carriers. According to an embodiment the fastener is a rack, and the stage has a three-dimensional shape which forms a counterpart to the rack comprising one or more carriers. It thereby allows an accurate positioning of the rack. In another embodiment, the fastener is a rack holding several standard microscope slides with FFPE tissue samples arranged on them. According to an embodiment, the fastener is a rack which may be placed on the stage or removed from the stage manually or by a robotic arm, which may be part of an automated laboratory system.

The light source according to the invention may be an emitter of light or other waves or particles. According to the invention the waves emitted may be of any wavelength, e.g., visible light and electromagnetic radiation including ultraviolet (UV) and infrared (IR).

In an embodiment, the emitted waves or particles are bundled in one direction or focused on an area of interest on the biologic specimen.

The shape of the light to emit from the light source according to the invention may be point, line, rectangle, or circle (e.g., ring light or segment thereof), or any other geometry. The light distribution according to the invention may be spherical, hemispherical, spot, photometric web, or any other pattern of light distribution.

The light source is placed at a first angle (α) relative to the plane. This angle (α) according to the invention may be optimized for a given biologic tissue or cells. For a thin layer of cells or tissue, shadowing is negligible, and therefore a point source or a longitudinal radiator may be the light source of choice. The light source generating an incident light targeting the biologic tissue or cells according to the invention is arranged on the same side of the plane formed by the stage as is the biologic tissue or cells. The light source is placed at a first angle (α) relative to the plane of 10 to 85 degrees. In an embodiment the light source is placed at a first angle (α) relative to the plane of 55 to 85 degrees. In an embodiment, the light source is placed at a first angle (α) relative to the plane of 65 to 75 degrees. In another embodiment, the light source is placed at a first angle (α) relative to the plane of 70 degrees.

In an embodiment according to the invention, the first angle (α) relative to the plane is smaller than the second angle (β) relative to the plane. In an embodiment, the first angle (α) relative to the plane is larger than the second angle (β) relative to the plane. In another embodiment, both angles (αand β) relative to the plane are equal.

In an embodiment according to the invention, the difference between the first angle (α) and the second angle (β) is larger than 5 degrees and smaller than 45 degrees.

In an embodiment, the light source and the camera are arranged such that they are both at the same distance from the plane. In another embodiment the light source and the camera are arranged such that the light source is at a greater distance from the plane than the camera. In a further embodiment, the light source and the camera are arranged such that the camera is at a greater distance from the plane than the light source.

The camera for detecting diffuse reflection off the biologic tissue or cells according to the invention may be any device that can record the waves emitted by the light source according to the invention, which may be of any wavelength, e.g., visible light and electromagnetic radiation including ultraviolet (UV) and infrared (IR). It may be a photographic camera, a digital camera, an IR camera, a multi-band camera, or any other camera. In an embodiment according to the invention the camera is arranged such that its angle (β) relative to the plane is larger than the first angle (α).

In an embodiment, the camera is arranged perpendicular to the stage, i.e., the second angle (β) relative to the plane equals 90 degrees. This arrangement has the advantage that distracting optical effects as a result of the thickness of the carrier are minimized. Such distracting optical effects may occur when some of the diffuse reflection reaches the stage and is reflected by specular reflection. This can result in a second shifted image when the camera is placed at an angle (β) relative to the plane that is smaller than 90 degrees. It is possible to eliminate some of these distracting optical effects by merging the first and shifted second images by image processing and computational methods.

In an embodiment according to the invention the camera takes high-contrast images of biologic tissue or cells in an automated laboratory system.

An automated laboratory system according to the invention may be an automated pipetting system, an automated incubator for biologic tissue or cells, or any other automated system including growth or maintenance of biologic tissue or cells. Such an automated laboratory system may comprise a data processor for calculating the transformations needed to align serial sample slides with the reference slide. In an embodiment the automated laboratory system may be a system for performing FFPE extractions comprising a dark-field imaging system. Such a system according to the invention improves the contrast of the image and thereby the degree of details that may be recorded in the grayscale range. This allows the user to align serial sample slides, e.g., FFPE tissue samples, with a reference slide, e.g., a hematoxylin and eosin (H&E) stained tissue sample, through x-axis, y-axis, and angle transformations, for which all image data in the entire grayscale range is used.

In an alternative embodiment the automated laboratory system may be a system for imaging biologic tissue or cells, comprising a dark-field imaging system according to the invention. The light source and the camera may be mounted on the automated laboratory system. The automated laboratory system may comprise one or more moveable robotic arms. One or more robotic arms may be constructed such that a gripper or any other tool can manually or automatically be mounted. Alternatively or additionally, one or more robotic arms may comprise means for pipetting liquids. The light source and/or the camera may be mounted on one or more robotic arms.

In an embodiment the automated laboratory system comprises a data processor for calculating the transformations needed to align serial sample slides with a reference slide. The camera may therefore be connected to a central processing unit with one or more output devices and interfaces.

Although the foregoing invention has been described in some detail for the purpose of illustration, it will be obvious that changes and modifications may be practiced within the scope of the appended claims by those of ordinary skill in the art.

The solution according to the invention, at least in preferred embodiments, inter alia achieves the following advantages:

The specular reflection of the incident light leaves for the camera to detect only the diffuse reflection off the biologic tissue or cells, resulting in images with a vastly improved contrast. Furthermore, the diffuse light scattered away from the camera is specularly reflected back onto the biologic tissue or cells and after recurring scattering reaches the camera, resulting in a significant increase in the brightness of the sample to be analyzed. This further ameliorates the contrast of the image.

In addition, with the stage in direct contact with the carrier adjacent to the biologic tissue or cells, it is possible to melt paraffin of formalin fixed paraffin embedded (FFPE) tissue samples in a controlled fashion, and thereby further reduce light scattering around the specimen. This results in even more contrast and improved image quality. As a result, the elevated contrast and dark background of the image provides enough image detail in the entire grayscale range, which enables the alignment of serial sample slides with the reference slide through x-axis, y-axis, and angle transformations, or by any other mathematical transformation.

Another advantage comes from the simple construction with all parts being placed on one side of the stage. This allows for a compact space saving design when integrated e.g., in an automated laboratory system. In addition, where the light source and camera are placed above the stage, the risk for contamination of either of the two through e.g., splashing or dripping is significantly reduced.

Overall the dark-field imaging system according to the invention will consequently yield better dark-field images allowing more processes to be automated and therefore causing less work and lower operating costs.

Furthermore, advantages and conveniences of the invention result from the following description of embodiments based on the drawings.

### BRIEF DESCRIPTION OF THE DRAWINGS

The invention will be described in detail with respect to the drawings schematically depicting embodiments of the invention. These are for illustrative purposes only and are not to be construed as limiting. In detail:

FIG. 1 shows a schematic full sectional view of a dark-field imaging system according to the invention, and

FIG. 2 shows the relevant angles in a schematic full sectional view of a dark-field imaging system according to the invention, and

FIG. 3 shows a schematic full sectional view of an embodiment of a dark-field imaging system according to the invention with a platform comprising a carrier for holding biologic tissue or cells, and a specularly reflecting stage for supporting the carrier, and

FIG. 4a shows a detailed schematic sectional view of an embodiment of a dark-field imaging system with a specularly reflecting carrier, and

FIG. 4b shows a detailed schematic sectional view of another embodiment of a dark-field imaging system with a specularly reflecting carrier, and

FIG. 4c shows a detailed schematic sectional view of an embodiment of a dark-field imaging system with a specularly reflecting stage, and

FIG. 5 shows a schematic full sectional view of an embodiment of a dark-field imaging system according to the invention with a specularly reflecting stage and a heating/cooling unit, and

FIG. 6 shows a schematic full sectional view of an automated laboratory system comprising an embodiment of a dark-field imaging system according to the invention.

### DETAILED DESCRIPTION OF THE INVENTION

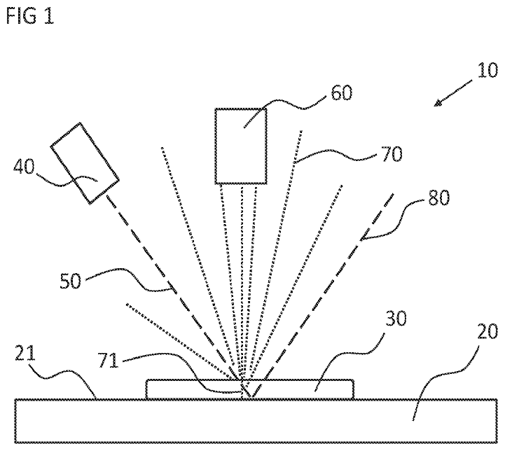

FIG. 1 shows a schematic full sectional view of a dark-field imaging system 10 according to the invention. The dark-field imaging system 10 shown here comprises a platform 20 constituting a plane 21 holding biologic tissue or cells 30. It further comprises a light source 40 generating an incident light 50 targeting the biologic tissue or cells 30, and a camera 60. The light source 40 is placed at a first angle (α) relative to the plane 21, and the camera 60 is placed at a second angle (β) relative to the plane 21. In this embodiment of the invention, the first angle (α) is smaller than the second angle (β), and the second angle (β) equals 90 degrees. In other words, in this embodiment the camera 60 is arranged perpendicular to the plane 21. The light source 40 generates an incident light 50, displayed as a dashed line, targeting the biologic tissue or cells 30. As shown the incident light 50 that is not reflected by the biologic tissue or cells 30 reaches the platform 20, which comprises a material that reflects the incident light 50 at least in part by specular reflection 80.

The camera 60 detects diffuse reflection 70 off the biologic tissue or cells 30, displayed as dotted lines. Diffuse reflection 70 is scattered in all directions, of which only a few representative light paths are shown. In the embodiment diffuse mirrored reflection 71 reaching the platform 20 is reflected at least in part by specular reflection 80, reflected mostly back onto the biologic tissue or cells 30. As the biologic tissue or cells 30 are typically only 5 to 10 μm in thickness, a considerable part of this specularly reflected light is emitted into the half-space formed by the plane 21 and comprising the light source 40, the camera 60, and the biologic tissue or cells 30. Some of this diffuse mirrored reflection 71, the one reaching the camera, is recorded together with the diffuse reflection 70 directly off the biologic tissue or cells 30.

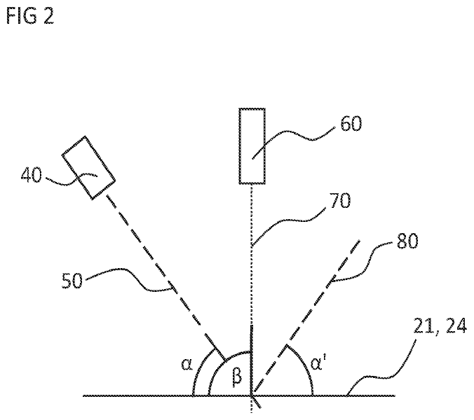

FIG. 2 shows the relevant angles in a schematic full sectional view of a dark-field imaging system 10 according to the invention. Shown here are the light source 40, the camera 60, the plane 21, the specularly reflecting surface 24, and the following light paths; the incident light 50 from the light source 40, its specular reflection 80 off the specularly reflecting surface 24, and the diffuse reflection 70 scattered off the biologic tissue or cells 30. The light source 40 is placed at a first angle (α) relative to the plane 21, or in this representation, relative to the specularly reflecting surface 24. The camera 60 is placed at a second angle (β) relative to the plane 21 and the specularly reflecting surface 24, respectively. In the embodiment according to the invention shown, the first angle (α) is smaller than the second angle (β). The angle alpha prime (α') is the angle of reflection of the incident light 50 producing the specular reflection 80 off the specularly reflecting surface 24. Therefore, the angle of reflection alpha prime (α') by definition equals the angle of incidence, which is the first angle (α).

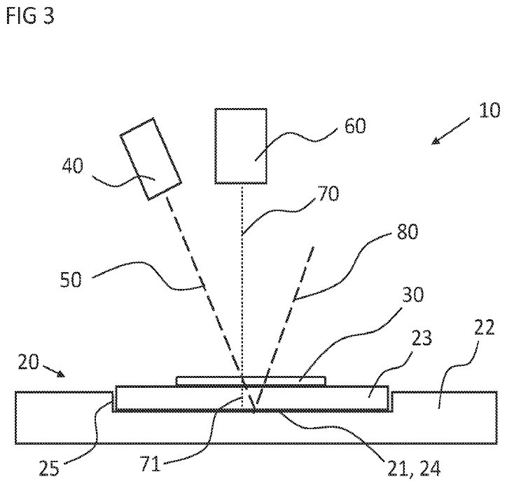

FIG. 3 shows a schematic full sectional view of an embodiment of a dark-field imaging system according to the invention with a platform 20 constituting a plane 21 forming two half-spaces, and comprising a carrier 23 for holding biologic tissue or cells 30, and a stage 22 for supporting the carrier 23. Shown is an exemplary embodiment with a specularly reflecting stage 22 comprising a fastener for holding one or more carriers 23 with biologic tissue or cells 30. The fastener 25 in the embodiment shown comprises one or more recesses with the dimensions of the one or more carriers 23, which may be one or more microscope slides.

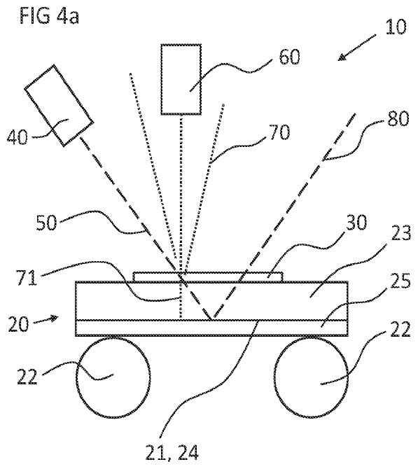

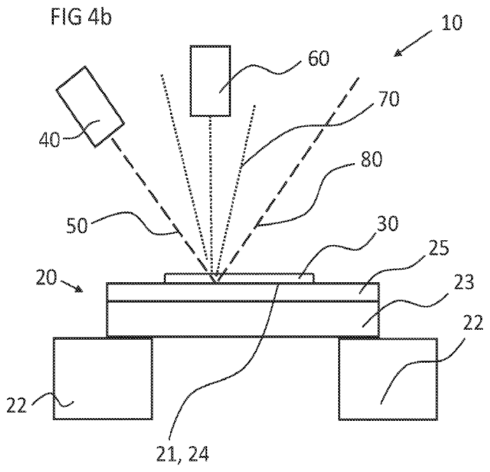

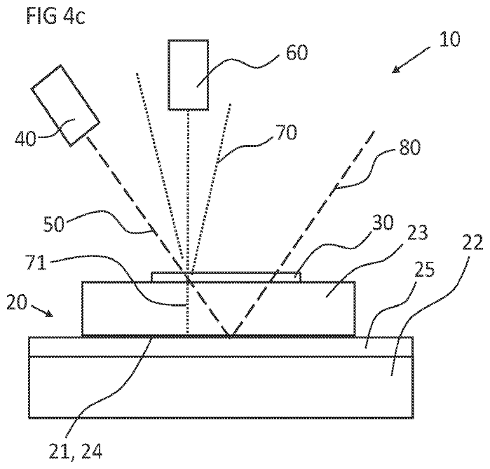

FIGS. 4a and 4b show detailed partly enlarged schematic sectional views of embodiments of a dark-field imaging system 10 with a platform 20 constituting a plane 21 forming two half-spaces, and comprising a specularly reflecting carrier 23 holding biologic tissue or cells 30, and a stage 22 for supporting the carrier 23. The specularly reflecting carrier 23 comprises a specularly reflecting material 25. As indicated here, the stage can be of any three-dimensional shape and can comprise one or more than one bodies, e.g., four spheres or two rods (FIG. 4a) or two or more beams (FIG. 4b), or one block (FIG. 4c).

Shown here are the light source 40, the stage 22, the carrier 23 comprising a specularly reflecting surface 24, and the biologic tissue or cells 30. The light source 40 generates an incident light 50, displayed as a dashed line, targeting the biologic tissue or cells 30, where it is scattered in all directions, i.e., reflected in a diffuse reflection 70, of which only a few representative light paths are displayed as dotted lines. The camera 60 detects some of the diffuse reflection 70 off the biologic tissue or cells 30. The fraction of the incident light 50 that is not reflected by the biologic tissue or cells 30 reaches the carrier 23 and is reflected specularly at the specularly reflecting surface 24 of the specularly reflecting material 25.

The carrier 23 in the embodiment according to the invention shown in FIG. 4a may comprise glass, polystyrene, tissue culture polystyrene, polypropylene, cyclic olefin copolymer, polymethylmethacrylate, or any other material transparent for the wavelength emitted by the light source 40, and is fused or coated on one side with a material that reflects the incident light 50 at least in part by specular reflection 80. As shown the carrier 23 comprises both a transparent material and a specularly reflecting material 25, the latter forming a specularly reflecting surface 24. In the embodiment shown in FIG. 4a, the specularly reflecting material 25 is fused to or coated on the side of the carrier 23 not facing the biologic tissue or cells 30. It is therefore not in contact with the biologic tissue or cells 30. Nevertheless, the specularly reflecting surface 24 faces the biologic tissue or cells 30.

The carrier 23 in the embodiment according to the invention shown in FIG. 4b reflects the remaining incident light 50 at is surface, which is the surface of the specularly reflecting material 25. The specularly reflecting material 25 may comprise any material that reflects the incident light 50 at least in part by specular reflection 80. Here the specularly reflecting material 25 is fused to or coated on the side of the carrier 23 facing the biologic tissue or cells 30 and the specularly reflecting surface 24 is in contact with the biologic tissue or cells 30. Alternatively, the entire carrier 23 may consists of a material that reflects the incident light 50 at least in part by specular reflection 80.

FIG. 4c shows a detailed partly enlarged schematic sectional view of an embodiment of a dark-field imaging system 10 with a platform 20 constituting a plane 21 forming two half-spaces, and comprising a transparent carrier 23 holding biologic tissue or cells 30, and a specularly reflecting stage 22 for supporting the carrier 23. Shown here are the light source 40, the biologic tissue or cells 30, the carrier 23, and the stage 22 comprising a specularly reflecting material 25 forming a specularly reflecting surface 24. The light source 40 generates an incident light 50, displayed as a dashed line, targeting the biologic tissue or cells 30, where it is scattered in all directions, i.e., reflected in a diffuse reflection 70, of which only a few representative light paths are displayed as dotted lines. The camera 60 detects some of the diffuse reflection 70 off the biologic tissue or cells 30. The fraction of the incident light 50 that is not reflected by the biologic tissue or cells 30 reaches the carrier 23 and travels through the transparent carrier until it hits the specularly reflecting stage 22. In the embodiment shown the remaining incident light 50 is thereby reflected at least in part by specular reflection 80. Diffuse mirrored reflection 71 reaching the stage 22 is equally reflected at least in part by specular reflection 80. Some of this reflection, the one reaching the camera, is recorded together with the diffuse reflection 70 directly off the biologic tissue or cells 30.

The stage 22 in the embodiment according to the invention shown here reflects the remaining incident light 50 at is surface and may comprise any material that is fused or coated on the side facing the carrier with a material that reflects the incident light 50 at least in part by specular reflection 80. Alternatively, the entire stage 22 consists of a material that reflects the incident light 50 at least in part by specular reflection 80.

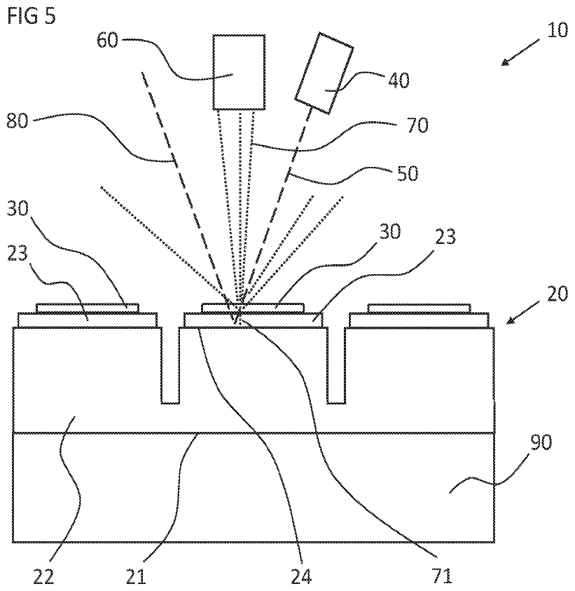

FIG. 5 shows a schematic full sectional view of an embodiment of a dark-field imaging system 10 according to the invention with a platform 20 constituting a plane 21 forming two half-spaces, and comprising a specularly reflecting stage 22, one or more carriers 23, and a heating/cooling unit 90. The heating/cooling unit 90 is in contact with the stage 22. In the embodiment shown, the specularly reflecting stage 22 is constructed as a temperature adapter, which channels and conducts the temperature from the heating/cooling unit 90 to the one or more carriers 23 with biologic tissue or cells 30. The temperature of the stage 22 can thereby be elevated to melt the paraffin of formalin fixed paraffin embedded (FFPE) tissue samples and as a result, the light scattering around the biologic tissue or cells 30 is mostly eliminated.

Shown are further a light source 40 generating an incident light 50 targeting the biologic tissue or cells 30, and a camera 60. The light source 40 is placed at a first angle (α) relative to the plane 21, and the camera 60 is placed at a second angle (β) relative to the plane 21. In the embodiment shown, the first angle (α) is smaller than the second angle (β), and the second angle (β) equals 90 degrees, i.e., in this embodiment the camera 60 is arranged perpendicular to the plane 21. The light source 40 generates an incident light 50, displayed as a dashed line, targeting the biologic tissue or cells 30. The incident light 50 that is not reflected by the biologic tissue or cells 30 reaches the platform 20, which comprises a material that reflects the incident light 50 at least in part by specular reflection 80.

The camera 60 detects diffuse reflection 70 off the biologic tissue or cells 30, displayed as dotted lines. Diffuse reflection 70 is scattered in all directions, of which only a few representative light paths are shown. In the embodiment diffuse mirrored reflection 71 reaching the platform 20 is reflected at least in part by specular reflection 80. Some of this diffuse mirrored reflection 71, the one reaching the camera, is recorded together with the diffuse reflection 70 directly off the biologic tissue or cells 30.

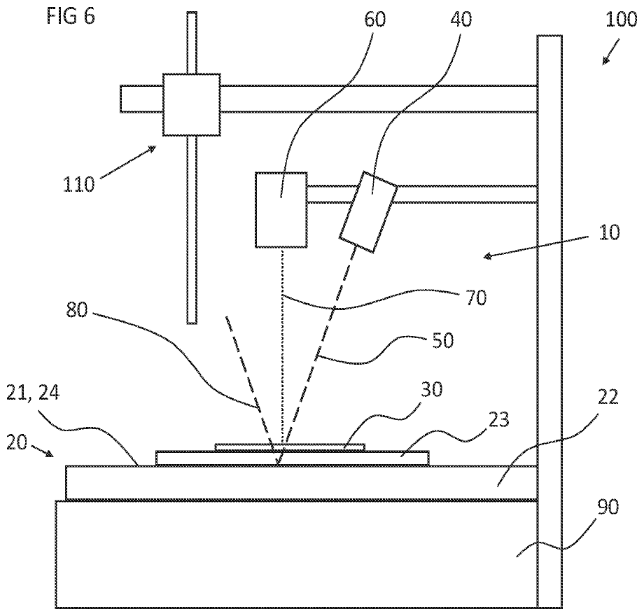

FIG. 6 shows a schematic full sectional view of an automated laboratory system 100 comprising an embodiment of a dark-field imaging system 10 according to the invention. Shown here are the light source 40 and the camera 60 mounted on the automated laboratory system 100, and the platform 20 constituting a plane 21 forming two half-spaces, and comprising a specularly reflecting stage 22, a carrier 23, and a heating/cooling unit 90. The heating/cooling unit 90 is in contact with the stage 22 and thereby allowing temperature adjustment. The specularly reflecting stage 22 may conduct the temperature from the heating/cooling unit 90 to the carrier 23 holding biologic tissue or cells 30. Also shown is the schematic representation of a moveable robotic arm 110. One or more robotic arms 110 may be constructed such that a gripper or any other tool can manually or automatically be mounted. Alternatively or additionally, the one or more robotic arms 110 may comprise means for pipetting liquids. The light source 40 and/or the camera 60 may be mounted on one or more robotic arms 110.

In the embodiment shown, the carrier 23 is transparent for the wavelength emitted by the light source 40, while the stage 22 comprises a material that reflects the incident light 50 at least in part by specular reflection 80. The specularly reflecting surface 24 in the embodiment shown is therefore at the surface of the stage 22 facing the carrier 23.

Also shown are the following light paths; the incident light 50 from the light source 40, its specular reflection 80 off the specularly reflecting surface 24, and the diffuse reflection 70 scattered off the biologic tissue or cells 30, which is captured by the camera 60. The light source 40 is placed at a first angle (α, not shown) relative to the plane 21, and the camera 60 is placed at a second angle (β, not shown) relative to the plane 21.

Incidentally it is also possible to implement the invention in a variety of variations in hereby shown examples and aspects of the invention highlighted above.

### LIST OF REFERENCE SIGNS

10 dark-field imaging system
20 platform
21 plane
22 stage
23 carrier
24 specularly reflecting surface
25 specularly reflecting material
30 biologic tissue or cells
40 light source
50 incident light
60 camera
70 diffuse reflection
71 diffuse mirrored reflection
80 specular reflection
90 heating/cooling unit
100 automated laboratory system
110 robotic arm

1. A dark-field imaging system (10) comprising:
a platform (20) for holding biologic tissue or cells (30), constituting a plane (21) forming two half-spaces;
a light source (40) generating an incident light (50) targeting the biologic tissue or cells (30); the light source (40) being placed at a first angle (a) relative to the plane (21); and a camera (60) to detect diffuse reflection (70) off the biologic tissue or cells (30), which is placed at a second angle (P) relative to the plane (21),
wherein the light source (40), the camera (60), and the biologic tissue or cells (30) are arranged in the same one of the two half-spaces formed by the plane (21);
wherein the incident light (50) that is not reflected by the biologic tissue or cells (30) reaches the platform (20); and
wherein the platform (20) comprises a specularly reflecting material (25) that reflects the incident light (50) at least in part by specular reflection (80).

2. The dark-field imaging system (10) according to claim 1, wherein the specularly reflecting material (25) of the platform (20) comprises a metal comprising a mirror-like surface, or an amorphous or non-crystalline solid comprising a mirror-like surface.

3. The dark-field imaging system (10) according to claim 1, wherein the specularly reflecting material (25) comprises chromium, aluminum, or silver.

4. The dark-field imaging system (10) according to claim 1, wherein the platform (20) comprises a carrier (23) for holding biologic tissue or cells (30), and a stage (22) for supporting the carrier (23).

5. The dark-field imaging system (10) according to claim 4, wherein the carrier (23) comprises a material transparent for the wavelength emitted by the light source (40), and the stage (22) comprises a specularly reflecting material (25) forming a specularly reflecting surface (24) facing the carrier (23).

6. The dark-field imaging system (10) according to claim 4, wherein the carrier (23) is a microscope slide, a cell culture chamber, flask, dish, or microplate.

7. The dark-field imaging system (10) according to claim 6, wherein the carrier (23) comprises a specularly reflecting material (25) forming a specularly reflecting surface (24) in contact with the biologic tissue or cells 30.

8. The dark-field imaging system (10) according to claim 6, wherein the carrier (23) comprises a specularly reflecting material (25) forming a specularly reflecting surface (24) facing the biologic tissue or cells 30 but not in contact with the biologic tissue or cells 30.

9. The dark-field imaging system (10) according to claim 1, wherein the light source (40) and the camera (60) are arranged such that they are both at the same distance from the plane (21).

10. The dark-field imaging system (10) according to claim 1, wherein the first angle (a) relative to the plane (21) is smaller than the second angle (P) relative to the plane (21), and wherein the difference between the first angle (a) and the second angle (ft ) is larger than 5 degrees and smaller than 45 degrees.

11. The dark-field imaging system (10) according to claim 1, wherein the light source (40) is placed at a first angle (a) relative to the plane (21) of 55 to 85 degrees.

12. The dark-field imaging system (10) according to claim 1, wherein the camera (60) is arranged perpendicular to the plane (21), the second angle (P) being 90°.

13. The dark-field imaging system (10) according to claim 1, wherein the platform (20) comprises a heating/cooling unit (90) in physical contact with the carrier (23), or in physical contact with the stage (22) conducting the temperature from the heating/cooling unit (90) to the carrier (23).

14. The dark-field imaging system (10) according to claim 1 in an application for interacting with areas of interest of biological samples in an automated laboratory system (100).

15. An automated laboratory system (100) comprising: a dark-field imaging system (10) according to claim 1.
